(19) United States
(12) Patent Application Publication
(10) Pub. No.: US 2005/0069276 A1
(43) Pub. Date: Mar. 31, 2005

(54) Deuterated pyrazolopyrimidinones and drugs containing said compounds

(76) Inventor: Rudolf-Giesbert Alken, Zepernick (DE)

Correspondence Address: KRIEGSMAN & KRIEGSMAN, 665 FRANKLIN STREET, FRAMINGHAM, MA, 01702, US

(21) Appl. No.: 10/494,914

(22) PCT Filed: Nov. 7, 2002
(86) PCT No.: PCT/DE02/04216

(30) Foreign Application Priority Data
Nov. 7, 2001 (DE) 101 55 018.9

(51) Int. Cl. C07D 487/04

(52) U.S. Cl. 385/147

(57) ABSTRACT

The invention concerns deuterated pyrazolopyrimidinones as well as pharmaceuticals containing these compounds. In addition, the invention concerns the use of deuterated pyrazolopyrimidinones for the inhibition of thrombocyte adhesion and aggregation, for the long-term increase of memory and learning functions, as well as for the treatment of cardiac and circulatory disorders, hypertonia, pulmonary hypertonia, erectile dysfunction and obstructive respiratory disorders such as, e.g., bronchial asthma. In addition, the invention discloses pharmaceutical compositions of deuterated pyrazolopyrimidinones as well as their physiological compatible salts for the inhibition of thrombocyte adhesion and aggregation, for the long-term increase of memory and learning functions, as well as for the treatment of cardiac and circulatory disorders, hypertonia, pulmonary hypertonia, erectile dysfunction and obstructive respiratory disorders such as, e.g., bronchial asthma, in addition to pharmaceutically compatible adjuvants and/or additives.

The invention concerns deuterated pyrazolopyrimidinones and pharmaceuticals containing these compounds.

Various pyrazolopyrimidinone derivatives are known to be effective and selective cGMP PDE5 inhibitors and are employed, among other things, for the treatment of cardiac and circulatory disorders, hypertonia and erectile dysfunction. A known representative of this substance class is sildenafil (U.S. Pat. No. 5,250,534 A1, EP 463,756 B1).

The object of the present invention is to prepare pyrazolopyrimidinones which have improved pharmacokinetic and/or pharmacodynamic properties when compared with the compounds already known.

It has now been found surprisingly that the deuterated pyrazolopyrimidinones according to the invention have essentially better pharmacokinetic and/or pharmacodynamic properties than the corresponding undeuterated compounds.

According to the invention the object is thus solved by the preparation of deuterated pyrazolopyrimidinones of the general formula I: 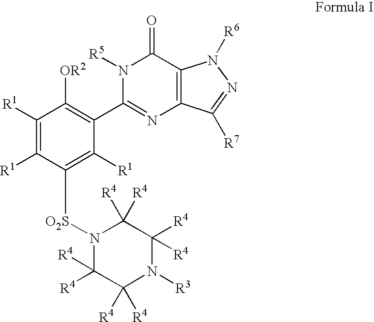
wherein R¹, independent of one another, is H or D, R² is C₁-C₃-alkyl, deuteroalkyl or perdeuteroalkyl, R³ is H, D, C₁-C₆-alkyl, deuteroalkyl or perdeuteroalkyl,
R⁴, independent of one another, is H or D, R⁵ is H or D, R⁶ represents H, D, C₁-C₃-alkyl, deuteroalkyl or perdeuteroalkyl, R⁷ is C₁-C₃-alkyl, deuteroalkyl or perdeuteroalkyl, and wherein at least one of the groups R¹ to R⁴ is deuterium or contains deuterium.

Deuterated pyrazolopyrimidinones of the general formula I are preferred, wherein R¹ is D, R² is C₁-C₃-alkyl, deuteroalkyl or perdeuteroalkyl, R³ is C₁-C₆-alkyl, deuteroalkyl or perdeuteroalkyl, R⁴, independent of one another, is H or D, R⁵ is H or D, R⁶ represents C₁-C₃-alkyl, deuteroalkyl or perdeuteroalkyl, and R⁷ is C₁-C₃-alkyl, deuteroalkyl or perdeuteroalkyl.

Deuterated pyrazolopyrimidinones of the general formula I are particularly preferred, wherein R¹, independent of one another, is H or D, R² indicates perdeuteroethyl, R³ is C₁-C₆-alkyl, deuteroalkyl or perdeuteroalkyl, R⁴, independent of one another, is H or D, R⁵is H or D, R⁶ represents C₁-C₃-alkyl, deuteroalkyl or perdeuteroalkyl, and R⁷ is C₁-C₃-alkyl, deuteroalkyl or perdeuteroalkyl.

Deuterated pyrazolopyrimidinones of the general formula I are especially preferred, wherein R¹, independent of one another, is H or D, R² is C₁-C₃-alkyl, deuteroalkyl or perdeuteroalkyl, R³ is trideuteromethyl, R⁴, independent of one another, is H or D, R⁵ is H or D, R⁶ represents C₁-C₃-alkyl, deuteroalkyl or perdeuteroalkyl, and R⁷ is C₁-C₃-alkyl, deuteroalkyl or perdeuteroalkyl.

Deuterated pyrazolopyrimidinones of the general formula I are advantageous, wherein R¹, independent of one another, is H or D, R² is C₁-C₃-alkyl, deuteroalkyl or perdeuteroalkyl, R³ is C₁-C₆-alkyl, deuteroalkyl or perdeuteroalkyl, R⁴ is D, R⁵ is H or D, R⁶ represents C₁-C₃-alkyl, deuteroalkyl or perdeuteroalkyl, and R⁷ is C₁-C₃-alkyl, deuteroalkyl or perdeuteroalkyl.

Deuterated pyrazolopyrimidinones of the general formula I are particularly advantageous, wherein R¹ is D, R² is C₁-C₃-alkyl, deuteroalkyl or perdeuteroalkyl, R³ is C₁-C₆-alkyl, deuteroalkyl or perdeuteroalkyl, R⁴, independent of one another, is H or D, R⁵ is D, R⁶ represents C₁-C₃-alkyl, deuteroalkyl or perdeuteroalkyl, and R⁷ is C₁-C₃-alkyl, deuteroalkyl or perdeuteroalkyl.

In addition, deuterated pyrazolopyrimidinones of the general formula I are advantageous, wherein R¹ is D, R² indicates perdeuteroethyl, R³ is C₁-C₆-alkyl, deuteroalkyl or perdeuteroalkyl, R⁴, independent of one another, indicates H or D, R⁵ is H or D, R⁶ represents trideuteromethyl and R⁷ is C₁-C₃-alkyl, deuteroalkyl or perdeuteroalkyl.

Deuterated pyrazolopyrimidinones of the general formula I are especially advantageous, wherein R¹ is D, R² indicates perdeuteroethyl, R³ is C₁-C₆-alkyl, deuteroalkyl or perdeuteroalkyl, R⁴, independent of one another, indicates H or D, R⁵ is H or D, R⁵ represents C₁-C₃-alkyl, deuteroalkyl or perdeuteroalkyl, and R⁷ is perdeutero-n-propyl.

According to the invention, the object is solved by the preparation of pyrazolopyrimidinones of general formula I, namely
5-[2-d5-ethoxy-5-(4-methylpiperazine-1-sulfonyl)phenyl]-1-methyl-3-n-propyl-1,6-dihydropyrazolo-[4,3-d]-pyrimidin-7-one,
5-[2-d5-ethoxy-5-(4-methylpiperazine-1-sulfonyl)-3,4,6-trideuterophenyl]-1-methyl-3-n-propyl-1,6-dihydropyrazolo-[4,3-d]-pyrimidin-7-one,
5-[2-d5-ethoxy-5-(4-trideuteromethylpiperazine-1-sulfonyl)-3,4,6-trideuterophenyl]-1-methyl-3-n-propyl-1,6-dihydropyrazolo-[4,3-d]-pyrimidin-7-one,
5-[2-d5-ethoxy-5-(d11-4-methylpiperazine-1-sulfonyl)-3,4,6-trideuterophenyl]-1-methyl-3-n-propyl-1,6-dihydropyrazolo-[4,3-d]-pyrimidin-7-one,
5-[2-d5-ethoxy-5-(d11-4-methylpiperazine-1-sulfonyl)-3,4,6-trideuterophenyl]-1-trideuteromethyl-3-n-propyl-1,6-dihydropyrazolo-[4,3-d]-pyrimidin-7-one,
5-[2-d5-ethoxy-5(d11-4-methylpiperazine-1-sulfonyl)-3,4,6-trideuterophenyl]-1-trideuteromethyl-3-n-propyl-1,6-dihydropyrazolo-[4,3-d]-6D-pyrimidin-7-one,
5-[2-d5-ethoxy-5-(d11-4-methylpiperazine-1-sulfonyl)-3,4,6-trideuterophenyl]-1-trideuteromethyl-3-n-d7-propyl-1,6-dihydropyrazolo-[4,3-d]-pyrimidin-7-one,
5-[2-d5-ethoxy-5-(d11-4-methylpiperazine-1-sulfonyl)-3,4,6-trideuterophenyl]-1-trideuteromethyl-3-n-d7-propyl-1,6-dihydropyrazolo-[4,3-d]-6D-pyrimidin-7-one,
5-[2-ethoxy-5-(4-trideuteromethylpiperazine-1-sulfonyl)phenyl]-1-methyl-3-n-propyl-1,6-dihydropyrazolo-[4,3-d]-pyrimidin-7-one,
5-[2-ethoxy-5-(4-trideuteromethylpiperazine-1-sulfonyl)phenyl]-1-trideuteromethyl-3-n-propyl-1,6-dihydropyrazolo-[4,3-d]-pyrimidin-7-one,
5-[2-ethoxy-5-(4-trideuteromethylpiperazine-1-sulfonyl)phenyl]-1-methyl-3-n-d7-propyl-1,6-dihydropyrazolo-[4,3-d]-pyrimidin-7-one,
5-[2-ethoxy-5-(4-trideuteromethylpiperazine-1-sulfonyl)phenyl]-1-methyl-3-n-propyl-1,6-dihydropyrazolo-[4,3-d]-6D-pyrimidin-7-one,
5-[2-ethoxy-5-(d11-4-methylpiperazine-1-sulfonyl)phenyl]-1-trideuteromethyl-3-n-propyl-1,6-dihydropyrazolo-[4,3-d]-pyrimidin-7-one,
5-[2-ethoxy-5-(d11-4-methylpiperazine-1-sulfonyl)phenyl]-1-methyl-3-n-d7-propyl-dihydropyrazolo-[4,3-d]-pyrimidin-7-one,
5-[2-ethoxy-5-(d11-4-methylpiperazine-1-sulfonyl)phenyl]-1-trideuteromethyl-3-n-d7-propyl-1,6-dihydropyrazolo-[4,3-d]-pyrimidin-7-one and
5-[2-ethoxy-5-(d11-4-methylpiperazine-1-sulfonyl)phenyl]-1-trideuteromethyl-3-n-d7-propyl-1,6-dihydropyrazolo-[4,3-d]-6D-pyrimidin-7-one,
5-[2-d5-ethoxy-5-(piperazine-1-sulfonyl)phenyl]-1-methyl-3-n-propyl-1,6-dihydropyrazolo-[4,3-d]-pyrimidin-7-one,
5-[2-d5-ethoxy-5-(piperazine-1-sulfonyl)-3,4,6-trideuterophenyl]-1-methyl-3-n-propyl-1,6-dihydropyrazolo-[4,3-d]-pyrimidin-7-one,
5-[2-d5-ethoxy-5-(2,2,3,3,5,5,6,6-octadeuteropiperazine-1-sulfonyl)-3,4,6-trideuterophenyl]-1-methyl-3-n-propyl-1,6-dihydropyrazolo-[4,3-d]-pyrimidin-7-one,
5-[2-d5-ethoxy-5-(2,2,3,3,5,5,6,6-octadeuteropiperazine-1-sulfonyl)-3,4,6-trideuterophenyl]-1-trideuteromethyl-3-n-propyl-1,6-dihydropyrazolo-[4,3-d]-pyrimidin-7-one,
5-[2-d5-ethoxy-5-(2,2,3,3,5,5,6,6-octadeuteropiperazine-1-sulfonyl)-3,4,6-trideuterophenyl]-1-trideuteromethyl-3-n-propyl-1,6-dihydropyrazolo-[4,3-d]-6D-pyrimidin-7-one,
5-[2-d5-ethoxy-5-(2,2,3,3 5,5,6,6-octadeuteropiperazine-1-sulfonyl)-3,4,6-trideuterophenyl]-1-trideuteromethyl-3-n-d7-propyl-1,6-dihydropyrazolo-[4,3-d]-pyrimidin-7-one,
5-[2-d5-ethoxy-5-(2,2,3,3,5,5,6,6-octadeuteropiperazine-1-sulfonyl)-3,4,6-trideuterophenyl]-1-trideuteromethyl-3-n-d7-propyl-1,6-dihydropyrazolo-[4,3-d]-6D-pyrimidin-7-one,
5-[2-ethoxy-5-(2,2,3,3,5,5,6,6-octadeuteropiperazine-1-sulfonyl)phenyl]-1-trideuteromethyl]-3-n-propyl-1,6-dihydropyrazolo-[4,3-d]-pyrimidin-7-one,
5-[2-ethoxy-5-(2,2,3,3,5,5,6,6-octadeuteropiperazine-1-sulfonyl)phenyl]-1-methyl-3-n-d7-propyl-1,6-dihydropyrazolo-[4,3-d]-pyrimidin-7-one,
5-[2-ethoxy-5-(2,2,3,3,5,5,6,6-octadeuteropiperazine-1-sulfonyl)phenyl]-1-trideuteromethyl-3-n-d7-propyl-1,6-dihydropyrazolo-[4,3-d]-pyrimidin-7-one,
5-[2-ethoxy-5-(2,2,3,3,5,5,6,6-octadeuteropiperazine-1-sulfonyl)phenyl]-1-trideuteromethyl-3-n-d7-propyl-1,6-dihydropyrazolo[4,3-d]-6D-pyrimidin-7-one,
5-[2-d5-ethoxy-5-(d9-piperazine-1-sulfonyl)-3,4,6-trideuterophenyl]-1-methyl-3-n-propyl-1,6-dihydropyrazolo-[4,3-d]-pyrimidin-7-one,
5-[2-d5-ethoxy-5-(d9-piperazine-1-sulfonyl)-3,4,6-trideuterophenyl]-1-trideuteromethyl-3-n-propyl-1,6-dihydropyrazolo-[4,3-d]-pyrimidin-7-one,
5-[2-d5-ethoxy-5-(d9-piperazine-1-sulfonyl)-3,4,6-trideuterophenyl]-1-trideuteromethyl-n-propyl-1,6-dihydropyrazolo-[4,3-d]-6D-pyrimidin-7-one,
5-[2-d5-ethoxy-5-(d9-piperazine-1-sulfonyl)-3,4,6-trideuterophenyl]-1-trideuteromethyl-n-d7-propyl-1,6-dihydropyrazolo-[4,3-d]-pyrimidin-7-one,
5-[2-d5-ethoxy-5-(d9-piperazine-1-sulfonyl)-3,4,6-trideuterophenyl]-1-trideuteromethyl-3-n-d7-propyl-1,6-dihydropyrazolo-[4,3-d]-6D-pyrimidin-7-one,
5-[2-ethoxy-5-(d9-piperazine-1-sulfonyl)phenyl]-1-trideuteromethyl-3-n-propyl-1,6-dihydropyrazolo-[4,3-d]-pyrimidin-7-one,
5-[2-ethoxy-5-(d9-piperazine-1-sulfonyl)phenyl]-1-methyl-3-n-d7-propyl-1,6-dihydropyrazolo-[4,3-d]-pyrimidin-7-one,
5-[2-ethoxy-5-(d9-piperazine-1-sulfonyl)phenyl]-1-trideuteromethyl-3-n-d7-propyl-1,6-dihydropyrazolo-[4,3-d]-pyrimidin-7-one,
5-[2-ethoxy-5-(d9-piperazine-1-sulfonyl)phenyl]-1-trideuteromethyl-3-n-d7-propyl-1,6-dihydropyrazolo-[4,3- d]-6D-pyrimidin-7-one.

The use of the deuterated pyrazolopyrimidinones according to the invention as well as their physiologically compatible salts is preferred for the inhibition of thrombocyte adhesion and aggregation, for the long-term increase of memory and learning functions, as well as for the treatment of cardiac and circulatory disorders, hypertonia, pulmonary hypertonia, erectile dysfunction and obstructive respiratory disorders such as, e.g., bronchial asthma.

The use of the deuterated pyrazolopyrimidinones according to the invention as well as their physiologically compatible salts is particularly preferred for the production of pharmaceuticals for the inhibition of thrombocyte adhesion and aggregation, for the long-term increase of memory and learning functions, as well as for the treatment of cardiac and circulatory disorders, hypertonia, pulmonary hypertonia, erectile dysfunction and obstructive respiratory disorders such as, e.g., bronchial asthma.

Pharmaceutical compositions are particularly preferred, which contain the deuterated pyrazolopyrimidinones according to the invention as well as their physiologically compatible salts for the inhibition of thrombocyte adhesion and aggregation, for the long-term increase of memory and learning functions, as well as for the treatment of cardiac and circulatory disorders, hypertonia, pulmonary hypertonia, erectile dysfunction and obstructive respiratory disorders such as, e.g., bronchial asthma,in addition to pharmaceutically compatible adjuvants and/or additives.

The deuterated pyrazolopyrimidinones according to the invention are produced on the basis of production processes for undeuterated compounds.

The synthesis of analogous undeuterated pyrazolopyrimidinones is described, for example, in U.S. Pat. No. 5,250,534 A1, EP 463,756 B1, EP 994,115 A2 and EP 812,845 B1. The methods conducted are particularly distinguished at the time point of cyclization to the pyrimidinone system relative to the introduction of the piperazine group.

The synthesis described in EP 812,845 B1 surpasses the other methods in terms of yield and purity of the products. This synthesis starts with a substituted pyrazole, which is coupled to an ethoxybenzoic acid substituted by methylpiperazine and is cyclized in the last step to pyrazolopyrimidinone.

The deuterated pyrazolopyrimidinones according to the invention are synthesized on the basis of this patent with respect to the reaction pathway, wherein, if necessary, the reaction conditions have been changed in order to avoid an H/D back exchange.

For the synthesis of the compounds according to the invention, one starts with 3-n-propylpyrazole 5-carboxylic acid ethyl ester, the production of which is analogous to Seki et al. [Chem. Pharm. Bull., 32 (4), pp. 1568-1577, 1984]. One starts with the corresponding deuterated precursors for the synthesis of the derivative deuterated in position 3.

This pyrazole is converted by N-methylation analogous to U.S. Pat. No. 5,250,534 or EP 463,756 with dimethyl sulfate or deuterated dimethyl sulfate into the optionally deuterated 1-methyl-3-n-propylpyrazole 5-carboxylic acid ester. The ester hydrolysis of this compound is produced in an acidic manner in the presence of a deuterated carboxylic acid ester, with the use of deuterium chloride solution. The undeuterated carboxylic acid ester is hydrolyzed in an alkaline manner analogous to U.S. Pat. No. 5,250,534.

A mixture of fuming nitric acid and fuming sulfuric acid can be used for nitrating pyrazole carboxylic acids, as described in U.S. Pat. No. 5,250,534 or EP 463,756. In one example of embodiment of the invention, the nitrating of position 1 of the deuterated compound under mild conditions by means of nitric acid in the presence of ammonium heptamolybdate is described (Sana et al., Chem. Lett., pp. 48-49, 2000).

The optionally deuterated 1-methyl-4-nitro-3-n-propylpyrazole 5-carboxylic acid is converted into the 5-carboxamide by reaction with thionyl chloride and ammonium hydroxide solution (U.S. Pat. No. 5,250,534 or EP 463,756). From this, one obtains the 4-amino-1-methyl-3-n-propylpyrazole 5-carboxamide by reduction of the nitro group, wherein, for the reduction of a deuterated pyrazole analogous to Ram et al. [Tetrahedron Lett., Vol. 25 (32), pp. 3415-3418, 1984], the reaction is conducted with Pd/C in the presence of ammonium formate at room temperature.

In the second part of the synthesis, analogous to EP 812,845 B1, 2-ethoxybenzoic acid or deuterated 2-ethoxybenzoic acid is sulfochlorinated in position 5 and then reacted with optionally deuterated 4-methylpiperazine, so that one obtains 2-ethoxy-5-(4-methylpiperazinesulfonyl)benzoic acid, or, with the use of deuterated educts, the corresponding deuterated compound.

The perdeuterated piperazine derivatives used according to the invention can be prepared analogously to known instructions for preparing the undeuterated compounds (U.S. Pat. No. 2,905,673, DE 2,205,597, DE 3,836,781).

3,3,5,5-Tetradeutero-1-methylpiperazine is prepared analogously to Shetty et al. [J. Labelled Compd. Radiopharm., Vol 18 (11), pp. 1633-1640, 1981].

For the synthesis of 2,2,6,6-tetradeutero-1-methylpiperazine and 2,2,6,6-tetradeutero-1-(trideuteromethyl)piperazine, analogously to Dischino et al. [J. Labelled Compd. Radiopharm., Vol 25 (4), pp. 359-367, 1987], N-benzylimidinodiacetic acid is brought to reaction with urea and the 1-benzyl-3,5-piperazinedione that forms is reacted with LiAlD₄ to 1-benzyl-3,3,5,5-tetradeuteropiperazine. The latter is then reacted with methyl iodide or trideuteromethyl iodide, as a modification of the instructions, and then debenzylated.

The coupling of the pyrazole derivative with the substituted benzoic acid, which now follows, is conducted in the presence of N,N′-carbonyl diimidazole.

The cyclization of the system performed in the last reaction step is conducted in tert-butanol with the addition of potassium tert-butoxide. The reaction product is precipitated by addition of deuterium chloride solution and one isolates the deuterated pyrazolopyrimidones according to the invention with a deuteration degree of at least 98%.

Common physiologically compatible inorganic and organic acids are, for example, hydrochloric acid, hydrobromic acid, phosphoric acid, sulfuric acid, oxalic acid, maleic acid, fumaric acid, lactic acid, tartaric acid, malic acid, citric acid, salicylic acid, adipic acid and benzoic acid. Other usable acids, for example, are described in Fortschritte der Arzneimittelforschung, Vol. 10, pages 224-225, Birkhäuser Publishing Co., Basel and Stuttgart, 1966, and Journal of Pharmaceutical Sciences, Vol. 66, pages 1-5 (1977).

The acid addition salts are usually obtained in a way known in and of itself by mixing the free bases or their solutions with the corresponding acids or their solutions in an organic solvent, for example, a lower alcohol such as methanol, ethanol, n-propanol or isopropanol or a lower ketone such as acetone, methyl ethyl ketone or methyl isobutyl ketone or an ether such as diethyl ether, tetrahydrofuran or dioxane. For better crystal deposition, mixtures of the named solvents can also be used. In addition, physiologically compatible aqueous solutions of acid addition salts of the compounds used according to the invention can be prepared in an aqueous acidic solution.

The acid addition salts of the compounds according to the invention can be converted into the free bases in a way known in and of itself, e.g., with alkalis or ion exchangers. From the free bases, by reaction with inorganic or organic acids, in particular, those which are suitable for the formation of salts that can be employed therapeutically further salts are obtained. These or also other salts of the new compound, such as, e.g., the picrate, can serve also for the purification of the free base by converting the free base into a salt, separating the latter, and again releasing the base from the salt.

The subject of the present invention is also pharmaceuticals for oral, buccal, sublingual, rectal, subcutaneoous, intravenous or intramuscular application or for inhalation, which contain, in addition to the usual vehicle and dilution agents, a compound of the general formula I or its acid addition salt as the active ingredient.

The pharmaceuticals of the invention are produced in the known way with the usual solid or liquid vehicle substances or dilution agents and the usually used pharmaceutical-technical adjuvants corresponding to the desired type of application with a suitable dosage. The preferred preparations exist in a form of administration which is suitable for oral, buccal or sublingual application. Such administration forms include, for example, tablets, tablets for chewing, sucking, or coated tablets, (sugar-)coated pills, capsules, pills, powders, solutions or suspensions or slow-release forms.

Of course, parenteral preparations such as injection solutions are also considered. In addition, suppositories can also be named, for example, as preparations. Corresponding tablets can be obtained, for example, by mixing the active ingredient with known adjuvants, for example, inert dilution agents such as dextrose, sugar, sorbitol, mannitol, polyvinylpyrrolidone, bursting agents such as corn starch or alginic acid, binding agents such as starch or gelatins, lubricants such as magnesium stearate or talcum and/or agents for obtaining a slow-release effect, such as carboxypolymethylene, carboxymethylcellulose, cellulose acetate-phthalate or polyvinyl acetate. The tablets can also comprise several layers.

Correspondingly, (sugar-)coated pills can be produced by coating cores, which are produced analogously to the tablets, with the agents usually employed in coating these pills, for example, polyvinylpyrrolidone or shellac, gum arabic, talcum, titanium dioxide or sugar. The envelope of the pill may also consist of several layers, whereby the above-mentioned adjuvants for tablets may be used.

Solutions or suspensions containing the active ingredient used according to the invention may additionally contain agents that improve taste such as saccharin, cyclamate or sugar, as well as, e.g., flavorings such as vanilla or orange extract. They may additionally contain suspension adjuvants such as sodium carboxymethylcellulose or preservatives such as p-hydroxybenzoate. Capsules containing active ingredients may be produced, for example, by mixing the active ingredient with an inert vehicle such as milk sugar or sorbitol and encapsulating in gelatin capsules.

Suitable suppositories can be prepared, for example, by mixing with support agents provided for this purpose, such as neutral fats or polyethylene glycol or their derivatives.

The production of the pharmaceutical preparations according to the invention is known in and of itself and is described in handbooks known to the person skilled in the art, for example, Hager's Handbuch [Handbook] (5th) 2, 622-1045; List et al., Arzneiformenlehre [Teachings of Drug Forms], Stuttgart: Wiss. Verlagsges. [Scientific Publishing Co.] 1985; Sucker et al., Pharmazeutische Technologie [Pharmaceutical Technology], Stuttgart: Thieme 1991; Ullmann's Enzyklopädie [Encyclopedia] (5th) A 19, 241-271; Voigt, Pharmazeutische Technologie [Pharmaceutical Technology], Berlin: Ullstein Mosby 1995.

The following examples explain the invention.

### EXAMPLE 1

### Production of 1-trideuteromethyl-3-n-propylpyrazole 5-carboxylic acid ethyl ester

Analogous to the production of the undeuterated compound, 18.3 g of 3-n-propylpyrazole 5-carboxylic acid ethyl ester are brought to reaction with 13.5 g of d6-dimethyl sulfate for 2.5 hours at 90° C. The mixture is then dissolved in dichloromethane, washed with an aqueous sodium carbonate solution, the organic phase is separated and dried and, after removal of the solvent, the material is purified by column chromatography. 14.2 g of 1-trideuteromethyl-3-n-propylpyrazole 5-carboxylic acid ethyl ester is obtained as a colorless oil.

Yield: 71 %

Theoretical:

C, 60.28%; H, 9.61%; N, 14.06%

Experimental:

C, 60.35%; H, 9.70%; N, 14.05%

¹H-NMR (200 MHz, CDCl₃): (1.10 (3H, t); 1.35 (3H, t); 1.73 (2H, m); 4.21 (2H, q); 6.80 (1H, s).

### EXAMPLE 2

### Production of 4D-1-trideuteromethyl-3-n-propylpyrazole 5-deuterocarboxylic acid

The carboxylic acid ester is hydrolyzed in deuterium chloride solution by suspending 9.96 g of 1-trideuteromethyl-3-n-propylpyrazole 5-carboxylic acid ethyl ester in deuterium chloride solution and heating to reflux for 15 hours. After workup, 5.7 g of reaction product are isolated as ivory-colored crystals.

Yield: 66%

Melting point: 147-151° C.

Theoretical:

C, 55.47%; H, 9.89%; N, 16.17%

Experimental:

C, 55.60%; H, 9.82%; N, 16.12%

¹H-NMR (200 MHz, d6-DMSO): (0.87 (3H, t); 1.60 (2H, m); 2.49 (2H, t).

### EXAMPLE 3

### Production of 1-trideuteromethyl-4-nitro-3-n-propylpyrazole 5-carboxylic acid

The pyrazole carboxylic acid is nitrated by means of nitric acid in the presence of ammonium heptamolybdate. 8.66 g of 4D-1-trideuteromethyl-3-n-propyl-5-pyrazole deuterocarboxylic acid are dissolved in dichloromethane, and after addition of 3.15 ml of 70% nitric acid and 61.75 g of ammonium heptamolybdate, are heated to reflux for 6 hours. The reaction batch is filtered, the solvent is removed and the obtained solid substance is purified by column chromatography. 8.43 g of product are obtained as a white solid.

Yield: 78%

Melting point: 122-126° C.

Theoretical:

C, 44.44%; H, 6.52%; N, 19.44%

Experimental:

C, 44.37%; H, 6.49%; N, 19.38%

¹H-NMR (200 MHz, CDCl₃): δ 0.92 (3H, t); 1.65 (2H, m); 2.51 (2H, t); 10.6 (1H, s).

### EXAMPLE 4

### Production of 1-trideuteromethyl-4-nitro-3-n-propylpyrazole 5-carboxamide

In a way known in and of itself, 10.85 g of 1-trideuteromethyl-4-nitro-3-n-propylpyrazole 5-carboxylic acid are added to 50 ml of thionyl chloride and the mixture is heated to reflux for 3 hours. The excess thionyl chloride is distilled off in vacuum, the residue is taken up with acetone, and carefully poured into an ice-cooled aqueous ammonium hydroxide solution that is mixed with ice. The precipitated reaction product is filtered off and 7.85 g of the carboxamide are obtained as a light-yellow solid.

Yield: 73%

Melting point: 137-141° C.

Theoretical:

C, 44.64%; H, 7.02%, N, 26.03%

Experimental:

C, 44.72%; H, 7.08%; N, 26.10%

¹H-NMR (200 MHz, CDCl₃): δ 0.93 (3H, t); 1.64 (2H, m); 2.51 (2H, t); 6.70 (2H, s).

### EXAMPLE 5

### Production of 4-amino-1-trideuteromethyl-3-n-propylpyrazole 5-carboxamide

10.8 g of the nitro compound are dissolved in dry methanol under argon and 2.5 g of 10% Pd—C are added to the solution. The reaction batch is mixed with 14.5 g of anhydrous ammonium formate and stirred for 30 minutes at room temperature. The catalyst is filtered off and washed with dry methanol. The filtrate is concentrated, the residue is mixed with water and the product is shaken out in dichloromethane. The organic phase is concentrated after drying and one obtains 7.5 g of 4-amino-1-trideuteromethyl-3-n-propylpyrazole 5-carboxamide as a white solid.

Yield: 81%

Melting point: 96-98° C.

Theoretical:

C, 51.87%; H, 9.25%; N, 30.25%

Experimental:

C, 51.93%; H, 9.22%; N, 30.19%

¹H-NMR (200 MHz, CDCl₃): δ 0.93 (3H, t); 1.64 (2H, m); 2.51 (2H, t); 4.60 (2H, s); 6.70 (2H, s).

### EXAMPLE 6

### Production of 1,3,4,6-tetradeutero-5-chlorosulfonyl-2-d5-ethoxybenzoic acid

Analogous to the production method of the undeuterated compound, 17.7 g of molten d10-2-ethoxybenzoic acid are added while stirring to an ice-cooled mixture of 7.5 ml of thionyl chloride and 28.2 ml of chlorosulfonic acid, wherein the temperature of the reaction batch is maintained below 25° C. The reaction batch is stirred for 18 hours at room temperature, then carefully poured into an ice-water mixture and stirred for another hour. The product which precipitates is separated, dried, and recrystallized from a hexane/toluene mixture. One obtains 20.45 g of a light-yellow solid.

Yield: 75%

Melting point: 111-114° C.

Theoretical:

C, 39.63%; H, 6.28%;

Experimental:

C, 39.88%; H, 6.20%

¹³C-NMR (200 MHz, d6-DMSO): δ 15.10 (sept); 64.70 (quint); 114.20 (t); 118.30 (s); 128.4 (t); 131.70 (t); 136.20 (s); 171.30 (s).

### EXAMPLE 7

### Production of 2-d5-ethoxy-5-(4-d11-methylpiperazine-1-sulfonyl) 3,4,6-trideuterobenzoic acid

Production is conducted in a way known in and of itself by adding 25.8 g of d12-4-methylpiperazine at 10° C. to a suspension of 27.3 g of 1,3,4,6-tetradeutero-5-chlorosulfonyl-2-d5-ethoxybenzoic acid in 95 ml of water, while stirring. The temperature of the reaction batch is kept at 20° C during the addition. The solution is cooled to 10° C. and stirred for another 2 hours at this temperature. The precipitating solid is filtered off, washed with ice water and dried. One obtains 26.75 g of crude product, which is immediately further processed after removal of a sample which is used to ascertain the structure.

Yield: 77%

Melting point: 192-196° C.

Theoretical:

C, 48.39%; H, 11.30%; N, 8.06%

Experimental:

C, 48.27%; H, 11.25%; N, 8.00%

¹³C-NMR (200 MHz, CDCl3): δ 14.90 (sept); 39.20 (quint); 46.00-46.40 (m); 56.10-56.50 (m); 113.70 (t); 115.90 (s); 127.60 (t); 131.70 (t); 129.50 (s); 162.90 (s); 171.30 (s).

### EXAMPLE 8

### Production of 4-[2-d5-ethoxy-(4-d11-methylpiperazine-1-sulfonyl)-3,4,6-trideuterobenzamido]-1-trideuteromethyl-3-n-propylpyrazole 5-carboxamide

This compound is produced analogously to the production method for the undeuterated compound by mixing together 27.3 g of 2-d5-ethoxy-5-(4-d11-methylpiperazin-1-ylsulfonyl)-3,4,6-trideuterobenzoic acid with 17.9 g of N,N′-carbonyl diimidazole in ethyl acetate and bringing these to reaction for 30 minutes at 55° C. and then for 2 hours while heating to reflux. 16.7 g of 4-amino-1-trideuteromethyl-3-n-propylpyrazole 5-carboxamide are added to this reaction batch and the latter is stirred for 72 hours at room temperature. The solid that settles out is isolated. The product is further processed without another purification; a sample is removed only for ascertaining structure and this sample is recrystallized from aqueous methanol. One obtains 29.6 g of product.

Yield: 85%

Melting point: 202-205° C.

Theoretical:

C, 51.34%; H, 10.57%; N, 16.33%

Experimental:

C, 51.43%; H, 10.49%; N, 16.28%

¹³C-NMR (200 MHz, CDCl₃): δ 13.90-14.30 (m); 22.10 (quint); 25.40 (quint); 32.90 (sept); 39.00 (sept); 46.30-46.70 (m); 56.70-57.10 (m); 63.90 (quint); 115.20 (t); 120.10 (s); 122.60 (s); 125.70 (t); 130.80-131.20 (m); 141.30 (s); 158.7 (s); 164.20 (s); 171.10 (s).

### EXAMPLE 9

### Production of 5-[2-d5-ethoxy-5-(d11-4-methylpiperazine-1-sulfonyl)-3,4,6-trideuterophenyl]-1-trideuteromethyl-3-n-propyl-1,6-dihydropyrazolo-[4,3-d]-6D-pyrimidin-7-one

12.9 g of 4-[2-d5-ethoxy-(4-d11-methylpiperazin-1-ylsulfonyl)-3,4,6-trideutero-benzamido]-1-trideuteromethyl-3-n-propylpyrazole 5-carboxamide are suspended in a way known in and of itself in tert-butanol and mixed with 3.37 g of potassium tert-butoxide. The mixture is heated to reflux for 8 hours, cooled to room temperature, and mixed with water. The solution that forms is [treated] dropwise with aqueous deuterium chloride solution. The reaction product that settles out is granulated at pH 7 and 10° C. for 1 hour, then filtered off, washed with water and dried. 10.36 g of product are isolated.

Yield: 83%

Melting point: 186-188° C.

Theoretical:

C, 53.09%; H, 10.72%; N, 16.89%

Experimental:

C, 53.21%; H, 10.83%; N, 16.75%

¹³C-NMR (200 MHz, CDCl₃): δ 13.90-14.30 (m); 22.80 (quint); 25.20 (quint); 32.80 (sept); 39.00 (sept); 46.30-46.70 (m); 56.70-57.10 (m); 65.80 (quint); 106.50 (s); 115.20 (t); 119.30 (s); 124.00 (t); 128.30 (t); 131.60 (s); 134.30 (s); 148.50 (s); 161.30 (s); 163.20 (s); 171.80 (s).

1. Deuterated pyrazolopyrimidinones of the general formula I 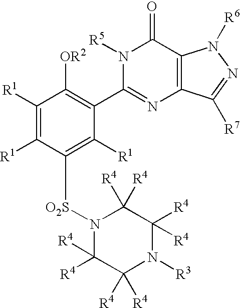
wherein R¹, independent of one another, is H or D,
R² is C₁-C₃-alkyl, deuteroalkyl or perdeuteroalkyl,
R³ is H, D, C₁-C₆-alkyl, deuteroalkyl or perdeuteroalkyl,
R⁴, independent of one another, indicates H or D,
R⁵ is H or D,
R⁶ represents H, D, C₁-C₃-alkyl, deuteroalkyl or perdeuteroalkyl,
R⁷ is C₁-C₃-alkyl, deuteroalkyl or perdeuteroalkyl, and wherein at least one of the groups R¹ to R⁴ is deuterium or contains deuterium.

2. Deuterated pyrazolopyrimidinones according to claim 1, further characterized in that
R¹ is D,
R² is C₁-C₃-alkyl, deuteroalkyl or perdeuteroalkyl,
R³ is C₁-C₆-alkyl, deuteroalkyl or perdeuteroalkyl,
R⁴, independent of one another, indicates H or D,
R⁵is H or D,
R⁶ represents C₁-C₃-alkyl, deuteroalkyl or perdeuteroalkyl, and
R⁷ is C₁-C₃-alkyl, deuteroalkyl or perdeuteroalkyl.

3. Deuterated pyrazolopyrimidinones according to claim 1, further characterized in that
R¹, independent of one another, is H or D,
R² indicates perdeuteroethyl,
R³ is C₁-C₆-alkyl, deuteroalkyl or perdeuteroalkyl,
R⁴, independent of one another, indicates H or D,
R⁵ is H or D,
R⁶ represents C₁-C₃-alkyl, deuteroalkyl or perdeuteroalkyl, and
R⁷ is C₁-C₃-alkyl, deuteroalkyl or perdeuteroalkyl.

4. Deuterated pyrazolopyrimidinones according to claim 1, further characterized in that
R¹, independent of one another, is H or D,
R² is C₁-C₃-alkyl, deuteroalkyl or perdeuteroalkyl,
R³ is trideuteromethyl,
R⁴, independent of one another, indicates H or D,
R⁵ is H or D,
R⁶ represents C₁-C₃-alkyl, deuteroalkyl or perdeuteroalkyl, and
R⁷ is C₁-C₃-alkyl, deuteroalkyl or perdeuteroalkyl.

5. Deuterated pyrazolopyrimidinones according to claim 1, further characterized in that
R¹, independent of one another, is H or D,
R² is C₁-C₃-alkyl, deuteroalkyl or perdeuteroalkyl,
R³ is C₁-C₆-alkyl, deuteroalkyl or perdeuteroalkyl,
R⁴ is D,
R⁵ is H or D,
R⁶ represents C₁-C₃-alkyl, deuteroalkyl or perdeuteroalkyl, and
R⁷ is C₁-C₃-alkyl, deuteroalkyl or perdeuteroalkyl.

6. Deuterated pyrazolopyrimidinones according to claim 1, further characterized in that
R¹ is D,
R² is C₁-C₃-alkyl, deuteroalkyl or perdeuteroalkyl,
R³ is C₁-C₆-alkyl, deuteroalkyl or perdeuteroalkyl,
R⁴, independent of one another, indicates H or D,
R⁵ is D,
R⁶ represents C₁-C₃-alkyl, deuteroalkyl or perdeuteroalkyl, and
R⁷ is C₁-C₃-alkyl, deuteroalkyl or perdeuteroalkyl.

7. Deuterated pyrazolopyrimidinones according to claim 1, further characterized in that
R¹ is D,
R² indicates perdeuteroethyl,
R³ is C₁-C₆-alkyl, deuteroalkyl or perdeuteroalkyl,
R⁴, independent of one another, indicates H or D,
R⁵is H or D,
R⁶ represents trideuteromethyl and
R⁷ is C₁-C₃-alkyl, deuteroalkyl or perdeuteroalkyl.

8. Deuterated pyrazolopyrimidinones according to claim 1, further characterized in that
R¹ is D,
R² indicates perdeuteroethyl,
R³ is C₁-C₆-alkyl, deuteroalkyl or perdeuteroalkyl,
R⁴, independent of one another, indicates H or D,
R⁵ is H or D,
R⁶ represents C₁-C₃-alkyl, deuteroalkyl or perdeuteroalkyl, and
R⁷ is perdeutero-n-propyl.

9. A compound selected from the group consisting of 5-[2-d5-Ethoxy-5-(4-methylpiperazine-1-sulfonyl)phenyl]-1-methyl-3-n-propyl-1,6-dihydropyrazolo-[4,3-d]-pyrimidin-7-one; 5-[2-d5-Ethoxy-5-(4-methylpiperazine-1-sulfonyl)-3,4,6-trideuterophenyl]-1-methyl-3-n-propyl-1,6-dihydropyrazolo-[4,3-d]-pyrimidin-7-one; 5-[2-d5-Ethoxy-5(4-trideuteromethylpiperazine-1-sulfonyl)-3,4,6-trideuterophenyl]-1-methyl-3-n-propyl-1,6-dihydropyrazolo-[4,3-d]-pyrimidin-7-one, 5-[2-d5-Ethoxy-5-(d11-4-methylpiperazine-1-sulfonyl)-3,4,6-trideuterophenyl]-1-methyl-3-n-propyl-1,6-dihydropyrazolo-[4,3-d]-pyrimidin-7-one: 5-[2-d5-Ethoxy-5-(d11-4-methylpiperazine-1-sulfonyl)-3,4,6-trideuterophenyl]-1-trideuteromethyl-3-n-propyl-1,6-dihydropyrazolo-[4,3-d]-pyrimidin-7-one; 5-[2-d5-Ethoxy-5-(d11-4-methylpiperazine-1-sulfonyl)-3,4,6-trideuterophenyl]-1-trideuteromethyl-3-n-propyl-1,6-dihydropyrazolo-[4,3-d]-6D-pyrimidin-7-one; 5-[2-d5-Ethoxy-5-(d11-4-methylpiperazine-1-sulfonyl)-3,4,6-trideuterophenyl]-1-trideuteromethyl-3-n-d7-propyl-1,6-dihydropyrazolo-[4,3-d]-pyrimidin-7-one: 5-[2-d5-Ethoxy-5-(d11-4-methylpiperazine-1-sulfonyl)-3,4,6-trideuterophenyl]-1-trideuteromethyl-3-n-d7-propyl-1,6-dihydropyrazolo-[4,3-d]-6D-pyrimidin-7-one; 5-[2-Ethoxy-5-(4-trideuteromethylpiperazine-1-sulfonyl)phenyl]-1-methyl-3-n-propyl-1,6-dihydropyrazolo-[4,3-d]-pyrimidin-7-one; 5-[2-Ethoxy-5-(4-trideuteromethylpiperazine-1-sulfonyl)phenyl]-1-trideuteromethyl-3-n-propyl-1,6-dihydropyrazolo-[4,3-d]-pyrimidin-7-one, 5-[2-Ethoxy-5-(4-trideuteromethylpiperazine-1-sulfonyl)phenyl]-1-methyl-3-n-d7-propyl-1,6-dihydropyrazolo-[4,3-d]-pyrimidin-7-one, 5-[2-Ethoxy-5-(4-trideuteromethylpiperazine-1-sulfonyl)phenyl]-1-methyl-3-n-propyl-1,6-dihydropyrazolo-[4,3-d]-6D-pyrimidin-7-one; 5-[2-Ethoxy-5-(d11-4-methylpiperazine-1-sulfonyl)phenyl-1-trideuteromethyl-3-n-propyl-1,6-dihydropyrazolo-[4,3-d]-pyrimidin-7-one; 5-[2-Ethoxy-5-(d11-4-methylpiperazine-1-sulfonyl)phenyl]-1-methyl-3-n-d7-propyl-1,6-dihydropyrazolo-[4,3-d]-pyrimidin-7-one; 5-[2-Ethoxy-5-(d11-4-methylpiperazine-1-sulfonyl)phenyl]-1-trideuteromethyl-3-n-d7-propyl-1,6-dihydropyrazolo-[4,3-d]-pyrimidin-7-one: 5-[2-Ethoxy-5-(d11-4-methylpiperazine-1-sulfonyl)phenyl-1-trideuteromethyl-3-n-d7-propyl-1,6-dihydropyrazolo-[4,3-d]-6D-pyrimidin-7-one; 5-[2-d5-Ethoxy-5-(piperazine-1-sulfonyl)phenyl]-1-methyl-3-n-propyl-1,6-dihydropyrazolo-[4,3-d]-pyrimidin-7-one: 5-[2-d5-Ethoxy-5-(piperazine-1-sulfonyl)-3,4,6-trideuterophenyl]-1-methyl-3-n-propyl-1,6-dihydropyrazolo-[4,3-d]-pyrimidin-7-one; 5-[2-d5-Ethoxy-5-(2,2,3,3,5,5,6,6-octadeuteropiperazine-1-sulfonyl)-3,4,6-trideuterophenyl]-1-methyl-3-n-propyl-1,6-dihydropyrazolo-[4,3-d]-pyrimidin-7-one, 5-[2-d5-Ethoxy-5-(2,2,3,3,5,5,6,6-octadeuteropiperazine-1-sulfonyl)-3,4,6-trideuterophenyl]-1-trideuteromethyl-3-n-propyl-1,6-dihydropyrazolo-[4,3-d]-pyrimidin-7-one: 5-[2-d5-Ethoxy-5-(2,2,3,3,5,5,6,6-octadeuteropiperazine-1-sulfonyl)-3,4,6-trideuterophenyl-1-trideuteromethyl-3-n-propyl-1,6-dihydropyrazolo-[4,3-d]-6D-pyrimidin-7-one, 5-[2-d5-Ethoxy-5-(2,2,3,3,5,5,6,6-octadeuteropiperazine-1-sulfonyl)-3,4,6-trideuterophenyl]-1-trideuteromethyl-3-n-d7-propyl-1,6-dihydropyrazolo-[4,3-d]-pyrimidin-7-one; 5-[2-d5-Ethoxy-5-(2,2,3,3,5,5,6,6-octadeuteropiperazine-1-sulfonyl)-3,4,6-trideuterophenyl]-1-trideuteromethyl-3-n-d7-propyl-1,6-dihydropyrazolo-[4,3-d]-6D-pyrimidin-7-one, 5-[2-Ethoxy-5-(2,2,3,3,5,5,6,6-octadeuteropiperazine-1-sulfonyl)phenyl]-1-trideuteromethyl-3-n-propyl-1,6-dihydropyrazolo-[4,3-d]-pyrimidin-7-one; 5-[2-Ethoxy-5-(2,2,3,3,5,5,6,6-octadeuteropiperazine-1-sulfonyl)phenyl]-1-methyl-3-n-d7-propyl-1,6-dihydropyrazolo-[4,3-d]-pyrimidin-7-one; 5-[2-Ethoxy-5-(2,2,3,3,5,5,6,6-octadeuteropiperazine-1-sulfonyl)phenyl]-1-trideuteromethyl-3-n-d7-propyl-1,6-dihydropyrazolo-[4,3-d]-pyrimidin-7-one; 5-[2-Ethoxy-5-(2,2,3,3,5,5,6,6-octadeuteropiperazine-1-sulfonyl)phenyl-1-trideuteromethyl-3-n-d7-propyl-1,6-dihydropyrazolo [4,3-d]-6D-pyrimidin-7-one; 5-[2-d5-Ethoxy-5-(d9-piperazine-1-sulfonyl)-3,4,6-trideuterophenyl]-1-methyl-3-n-propyl-1,6-dihydropyrazolo-[4,3-d]-pyrimidin-7-one; 5-[2-d5-Ethoxy-5-(d9-piperazine-1-sulfonyl)-3,4,6-trideuterophenyl]-1-trideuteromethyl-3-n-propyl-1,6-dihydropyrazolo-[4,3-d]-pyrimidin-7-one; 5-[2-d5-Ethoxy-5-(d9-piperazine-1-sulfonyl)-3,4,6-trideuterophenyl]-1-trideuteromethyl-3-n-propyl-1,6-dihydropyrazolo-[4,3-d]-6D-pydrimidin-7-one; 5-[2-d5-Ethoxy-5-(d9-piperazine-1-sulfonyl)-3,4,6-trideuterophenyl]-1-trideuteromethyl-3-n-d7-propyl-1,6-dihydropyrazolo-[4,3-d]-pyrimidin-7-one; 5-[2-d5-Ethoxy-5-(d9-piperazine-1-sulfonyl)-3,4,6-trideuterophenyl]-1-trideuteromethyl-3-n-d7-propyl-1,6-dihydropyrazolo-[4,3-d]-6D-pyrimidin-7-one; 5-[2-Ethoxy-5-(d9-piperazine-1-sulfonyl)phenyl]-1-trideuteromethyl-3-n-propyl-1,6-dihydropyrazolo-[4,3-d]-pyrimidin-7-one; 5-[2-Ethoxy-5-(d9-piperazine-1-sulfonyl)phenyl]-1-methyl-3-n-d7-propyl-1,6-dihydropyrazolo-[4,3-d]-pyrimidin-7-one; 5-[2-Ethoxy-5-(d9-piperazine-1-sulfonyl)phenyl]-1-trideuteromethyl-3-n-d7-propyl-1,6-dihydropyrazolo-[4,3-d]-pyrimidin-7-one; and 5-[2-Ethoxy-5-(d9-piperazine-1-sulfonyl)phenyl]-1-trideuteromethyl-3-n-d7-propyl-1,6-dihydropyrazolo-[4,3-d]-6D-pyrimidin-7-one.

10-44. (Canceled).

45. Use of the deuterated pyrazolopyrimidinones according to any one of claims 1 to 9 as well as their physiologically compatible salts for the inhibition of thrombocyte adhesion and aggregation, for the long-term increase of memory and learning functions, such as for the treatment of cardiac and circulatory disorders, hypertonia, pulmonary hypertonia, erectile dysfunction and obstructive respiratory disorders such as, e.g., bronchial asthma.

46. Use of the deuterated pyrazolopyrimidinones according to the invention according to any one of claims 1 to 9 as well as their physiologically compatible salts for the production of pharmaceuticals for the inhibition of thrombocyte adhesion and aggregation, for the long-term increase of memory and learning functions, as well as for the treatment of cardiac and circulatory disorders, hypertonia, pulmonary hypertonia, erectile dysfunction and obstructive respiratory disorders such as, e.g., bronchial asthma.

47. Pharmaceutical compositions containing deuterated pyrazolopyrimidinones according to any one of claims 1 to 9 as well as their physiologically compatible salts for the inhibition of thrombocyte adhesion and aggregation, for the long-term increase of memory and learning functions as well as for the treatment of cardiac and circulatory disorders, hypertonia, pulmonary hypertonia, erectile dysfunction and obstructive respiratory disorders such as, e.g., bronchial asthma, in addition to pharmaceutically compatible adjuvants and/or additives.
